(19) United States
(12) Patent Application Publication
(10) Pub. No.: US 2010/0016775 A1
(43) Pub. Date: Jan. 21, 2010

(54) Head trauma bandage and method

(76) Inventors: Michelle Cumming, Pacific Grove, CA (US); Mitchell Kastros, Carmel, CA (US)

Correspondence Address: MARCUS G THEODORE, PC, 466 SOUTH 500 EAST, SALT LAKE CITY, UT, 84102, US

(21) Appl. No.: 12/586,431

(22) Filed: Sep. 22, 2009

Related U.S. Application Data
(63) Continuation-in-part of application No. 12/156,512, filed on Jun. 2, 2008.

(51) Int. Cl. A61F 13/00

(52) U.S. Cl. 602/60

(57) ABSTRACT

An emergency head trauma bandage and method of use, which, when applied, applies minimal pressure to stop bleeding, doesn't compromise cervical spine immobilization, allows for fast and effective application of ice/cold packs to control intracranial/internal swelling, doesn't come apart during treatment and transport, and doesn't require a caregiver to re-wrap the dressing.

### RELATED APPLICATIONS

This applications is a continuation-in-part patent application of the patent application entitled “Head Trauma Cap Bandage”, Ser. No. 12/156,512 filed Jun. 2, 2008.

### BACKGROUND OF THE INVENTION

1. Field

The present invention relates to bandages and trauma treatment. In particular, it relates to a method of use and an emergency head trauma bandage, which is placed on the cranium of an injured patient with minimal movement of the neck and spine.

2. Description of Related Art

Various bandages are known in the art. Boukanov et al., U.S. Pat. No. 6,762,337 issued Jul. 13, 2004 discloses a multi-purpose pressure bandage for body wounds utilizing an expansion bladder, which inflates to compress an affixed bandage against an injured patient's wounds. The Boukanov et al. specifically states its system design is to provide a pressure dressing. To apply pressure, the device incorporates the use of a carbon dioxide gas container with an inlet valve for inflating a bladder in the bandage on site to apply additional pressure (resistance pressure or inflating to create pressure) to the wound to control bleeding.

The Boukanov et al. embodiment for head wounds has the compression bandage shaped like a cap to secure about the head. This cap configuration requires the head to be lifted for placement on head wounds, which may cause spinal damage. Also, sliding on of the cap may aggravate head wounds. The Boukanov et al head bandage configuration also has a bladder with a coextensive gauze bandage liner and a gas cartridge hidden in a pouch at a bottom edge. Elongated hook and loop straps extend diametrically from the bottom edge for securing the dome-shaped pressure bandage to a head injury. Once in place, the bandage is inflated to apply pressure to the wound. Although the application of pressure to control bleeding is taught to be the standard when treating soft tissue injuries, it is contraindicated with regard to bleeding associated with a head injury.

Boukanov et al.'s compression bandage is not suitable for head injuries. Head injuries are usually associated with intracranial swelling which causes excess pressure on the brain and towards the skull. Pressure treatment similar to Boukanov et al. applied to a head injury through compression compounds the problem of internal pressure to the brain and skull. Through this compression method the pressure applied by treating the injury creates even more pressure on the brain, and this can lead to a rapid deterioration of the condition of the patient. In addition, long-term and permanent brain damage can occur from the application of a pressure dressing, which, in the worst case, can lead to the death of the patient.

The standard of treatment for a head injury is to apply gentle pressure for controlling bleeding, and for applying ice to control intracranial swelling associated with head trauma. The idea of applying “gentle pressure” is to not exceed the amount of pressure being exerted inside the cranium resulting from head trauma.

The present invention discussed below is designed to be consistent with the standard for treating head injuries, and does not have any similarities with the Boukanov et al compression system with regard to its application. It does not have any features which create excess pressure, compromise cervical-spinal precautions or in any other way challenge the well-being of the patient with a head injury.

If the Boukanov et al. pressure regulation fails, it also may result in significant pressure, which can cause serious head injuries where intra cranial fluids build up causing the head to swell. In addition, if improperly inflated, circulation may be cut off. The bandage also suffers from compression problems if the gas container is empty, or fails to inflate the bladder. Under these circumstances, the Boukanov et al pressure bandage may aggravate the patient's head injuries. Further, if the Boukanov et al. bladder is pierced accidentally during emergency use, an ill fitting head wrap results.

Lundell et al., U.S. Design Patent, Des. 295,446, issued Apr. 26, 1988 is a head bandage protector that would require first conventionally wrapping the patent with bandages, which may compromise cervical spin immobilization depending on how the bandage wraps are administered.

Fye, U.S. Pat. No. 5,031,609, issued Jul. 16, 1991 is a postoperative compression bandage for the head, which would also require conventional bandaging before compression application; again possibly compromising cervical spine immobilization.

Neither Lundell et al, or Fye are combined bandages with a cover for rapid application in the field to avoid moving the neck or spine during emergency trauma applications.

Cited for general interest are: Sherwood, U.S. Pat. No. 5,044,031, issued Sep. 3, 1991 discloses passive warming articles for traumatized individuals suffering from hypothermia, shock or exposure. Kun, U.S. Pat. No. Des. 354,376, issued Feb. 14, 1995 discloses a head cooling cap. Hujar et al., U.S. Pat. No. 5,557,807 issued Sep. 24, 1996 discloses headwear including coolant means. Ameer, U.S. Pat. No. 6,228,041, issued May 8, 2001 discloses a lightweight portable scalp vibrating and hair growth stimulating device. Komachak, U.S. Publication No. US2007/0074326, dated Apr. 5, 2007, discloses a headgear with cooling device formed using a woven or non-woven material. Wang, U.S. Pat. No. 4,744,106, issued May 17, 1988 discloses an engineering cap with fan device structure for ventilation of the hard hat. Augustine et al., U.S. Pat. No. 5,860,292 issued Jan. 19, 1999 discloses an inflatable thermal blanket with head covering for convectively cooling the body. Robinson et al., U.S. Pat. No. 6,678,896, issued Jan. 20, 2004 discloses a sports towel. Ronquillo, U.S. Pat. No. 5,666,668 issued Sep. 16, 1997 discloses a cap with front size adjustment and rear flap. Dixon, U.S. Pat. No. 5,960,477 issued Oct. 5, 1999 discloses a hat with folded rim and visor. Dumas et al., U.S. Pub. No. 2005/0027227 published Feb. 3, 2005 discloses a disposable water resistant cover for medical applications. Reeves, U.S. Pat. No. 6,747,561 issued Jun. 8, 2004 discloses a bodily worn device, which provides for digital storage and retrieval of a user's medical records, drug prescriptions, medical history, organ donor instructions, and personal identification for use in an emergency or routine medical situation. Zucker et al., U.S. Publication No. US2005/0193491 published Sep. 8, 2005, discloses a pediatric emergency transport device. McKay, U.S. Pat. No. 5,305,470 issued Apr. 26, 1994 discloses a sportsband.

None of the above references provides an emergency head bandage, which doesn't compromise cervical spine immobilization, when applied, doesn't come apart during treatment and transport, and doesn't require a caregiver to re-wrap the dressing. The improved invention discussed below can be quickly applied as a bandage dressing to control bleeding and/or a device to secure ice packs to gently control intracranial pressure. These features can be used separately or in conjunction with a single application of the cap, depending on the medical needs of the patient with regard to head trauma. The invention described below provides such an invention and method of using it.

### SUMMARY OF THE INVENTION

The present invention comprises a flexible flat stretchable bandage with securable ends defining a slot structured to wrap around the head in a manner requiring minimal movement of the head. The securable ends, when secured, form a rimmed opening with enough stretch to fit about the cranium of the head securing about it just above the eyes and ears of a patient with a head trauma. The stretchable bandage allows the head to swell from cranial pressure, but provides sufficient contact with the wound to minimize bleeding. It is particularly suited for emergency field use, where rapid stabilization of a patient is required for transport. Minor cuts on the head often bleed heavily because the face and scalp have many blood vessels close to the surface of the skin. This bleeding is alarming, but often the injury is not severe and the bleeding will stop with modest pressure treatment. Head wounds encountered in the field, must be quickly covered to minimize bleeding to stabilize the patient for rapid transport for emergency treatment. Traditional bandaging requires multiple strips of gauze or sterile wrappings to be wound about the patient's head. This is often time consuming and requires the head to be repeatedly lifted or moved, which can aggravate spinal injuries.

The Boukanov type flexible cap bandage may aggravate open wounds when slipped on and may act as a compression bandage applying too much cranial pressure The present flexible trauma wrap bandage is quickly applied over the cranium in a manner, which does not compromise cervical spine immobilization, which can occur with conventional bandage wrapping. It not only stops bleeding, but it does not overly apply excessive pressure on the wound to stop circulation or aggravate intracranial pressure.

If head swelling occurs, the flexible trauma wrap bandage securable ends may be loosened and re-wrapped to prevent increasing intracranial pressure.

On the inside of the cap is attached a stretchable sterile dressing liner. The stretchable cap and dressing liner has enough stretch when placed on a patient to allow expansion and apply minimal pressure to control bleeding and hold the cap bandage in place.

It also has sufficient size, when secured, to accommodate swelling or hold an icepack/cold pack in place about the patent's head for closed dermal head injuries (hematomas) to control swelling. However, usually an external sleeve insert with pockets is attached around the wrap exterior into which the ice packs may be inserted to avoid contaminating the dressing liner or producing an ill fitting wrap bandage.

For larger heads, an extra fold may be included around the rim of the wrap, which is folded down if required to cover larger heads or additional areas of the skull. This extra fold also provides additional material around the rim to help secure the cap in place without straps where one cannot use straps to secure under the chin, where there is a jaw injury, or where the patient is regurgitating.

To secure the cap in place, an optional strap system may be employed to secure the cap bandage anchored under the chin, or about the head. This strap system may also be employed to secure ice packs within the exterior sleeves. In one embodiment, the strap system comprises a removable circumferential adjustable strap affixed to edges of the cap with hook and loop strips. This adjustable strap enables the cap to be secured in place by the brim. Alternatively, the adjustable strap ends may be secured to the cap brim to hold the cap in place about the head and under the chin. This adjustable strap, when secured, may be adjusted to place appropriate pressure to hold the cap about the head to control bleeding.

Other fasteners, such as snaps, hooks, buttons, etc. could be used to secure the strap ends, but these are more complicated to use in the field, and are more expensive and difficult to adjust.

The wrap and sterile dressing liner are both preferably constructed of absorbent cotton, which stretches approximately 20% to apply minimal resistance pressure to stop bleeding. It also has sufficient give to accommodate wound swelling. The bandage wrap and sterile dressing liner is packaged in a sterile wrap, which is removed just prior to use. It is inexpensive to manufacture, and may include a flexible liquid impervious cover, for outdoor use to provide weather protection. The bandage wrap may also be color coded to identify patients who have been given a medication or treatment, which requires special handling by emergency trauma teams. This is particularly important for field disasters requiring triage color categorization. In advanced triage systems, secondary triage is typically implemented by paramedics, battlefield medical personnel or by skilled nurses in the emergency departments of hospitals, and during disasters, where injured people are sorted into five categories (note; categories and color coordinates may vary according to regions and other requirements dictated by policy:

Black/Expectant (Monterey County, California category is “Morgue,” Pulseless/Non-Breathing)

They are so severely injured that they will die of their injuries, possibly in hours or days (large-body burns, severe trauma, lethal radiation dose), or in life-threatening medical crisis that they are unlikely to survive given the care available (cardiac arrest, septic shock, severe head or chest wounds); they should be taken to a holding area and given painkillers as required to reduce suffering.

Red/Immediate (same in Monterey County, California)

They require immediate surgery or other life-saving intervention, and have first priority for surgical teams or transport to advanced facilities; they “cannot wait” but are likely to survive with immediate treatment.

Yellow/Observation (Monterey, Calif. category is “Delayed,” Serious, Non-Life Threatening)

Their condition is stable for the moment but requires watching by trained persons and frequent re-triage, will need hospital care (and would receive immediate priority care under “normal” circumstances).

Green/Wait (walking wounded) (Monterey County, California category is “Minor”)

They will require a doctor's care in several hours or days but not immediately, may wait for a number of hours or be told to go home and come back the next day (broken bones without compound fractures, many soft tissue injuries).

White/Dismiss (walking wounded)

They have minor injuries; first aid and home care are sufficient, a doctor's care is not required. Injuries are along the lines of cuts and scrapes, or minor burns.

By color coding the bandage wraps by attaching triage tags to them or actually employing different colored caps, traumatized patients can quickly be directed for appropriate care.

As the trauma bandage wrap is a one-piece dressing and bandage, it is designed for simple, safe and quick application to the patient's head to control bleeding while minimizing movement to the patient's head. The biggest challenge in treating a head injury with bleeding is to minimize movement of the patient's head while effectively applying a dressing which will treat the wound and remain secure and intact on the patient's head. In any situation involving a head injury, with or without bleeding, there is also the chance of injury to the neck, back and spinal column. While treating the patient it is extremely important to minimize any action which will cause the head to move, possibly resulting in further injury to the spinal region. Protocols for the treatment of head injuries dictate caregivers to apply a cervical collar around the patient's neck and then secure the patient to a backboard in order to protect the spine. In the emergency medical field the trauma cap bandage will be applied and secured to the patient's head by one caregiver while a second caregiver maintains cervical spinal immobilization on the patient's head according to protocol, either before or after the patient is placed on the backboard. The proper application of the trauma cap wrap minimizes head and neck movement, which reduces the chances of cervical-spinal compromise to the patient.

With traditional methods of treating head trauma, a separate dressing is applied to the wound followed by a wrap bandage which is wound in such a way as to secure the dressing to the wound. This method has its drawbacks as, based on the location of the wound on the head plus other challenges such as hair thickness, possible head movement etc., it is often difficult to secure the bandage. This results in the bandage slipping off of the patient's head and the need to re-apply a new dressing. In situations involving major head trauma, this can be critical in terms of blood loss, head movement, spinal column compromise and extended on-scene time.

The trauma bandage wrap is capable of being applied in such a way to quickly, safely and effectively cover and secure whichever part of the head needs protecting. When placed in position, it covers the top of the head, or the extra fold extended to cover the sides and back of the head, which are the areas which cause challenges using traditional treatment methods. At whichever point on the patient's head the extension of the trauma bandage wrap stops, it can then be secured with the built-in Velcro straps.

The invention is thus particularly suited for emergency treatment of accident victims with head wounds. These are quickly bandaged before patient transport, thereby reducing triage time. This allows the patient to be more rapidly transported to a hospital where the cap bandage is quickly removed for examination and the wound treated.

An optional water resistant cover may be included to cover the exterior of the trauma bandage wrap where it is likely that the wrap and sterile dressing will be used in inclement weather to keep the patent's head wound dry.

The head trauma bandage wrap is thus readily slid onto the head of a traumatized patient in the field. It is particularly suited to be placed in a manner to not interfere with cervical spine immobilization of an immobilized patient with spinal or neck injuries. As the invention is of one piece construction, it will not come apart during treatment or transport. It is fast and easy to apply to not only apply gentle direct pressure to a head wound, but also to control the bleeding to enable other treatment of the patient to be completed. If bleeding is profuse, additional dressings may be inserted beneath the bandage wrap to absorb and control bleeding.

The invention thus provides an emergency head bandage, which doesn't compromise cervical spine immobilization, when applied, doesn't come apart during treatment and transport, and doesn't require a caregiver to re-wrap the dressing. The invention described below provides such an invention.

### DESCRIPTION OF THE DRAWINGS

FIG. 1 is a front view of a preferred embodiment of the invention.

FIG. 2 is a rear view of the embodiment shown in FIG. 1.

FIG. 3 is a bottom view of the inside of the embodiment shown in FIG. 1.

FIG. 4 is perspective full figure view of the embodiment shown in FIG. 1.

FIG. 5 is a perspective of another preferred embodiment of the invention.

FIG. 6 is another perspective of the embodiment shown in FIG. 5.

### DESCRIPTION OF THE ILLUSTRATED EMBODIMENTS

FIG. 1 illustrates a front view of a preferred embodiment of the invention 10, shown affixed about the head of a patient. It comprises a stretchable flexible wrap 10 with opposing releasable fastening ends 11 separated by a slit 11a shown in FIG. 3 forming an adjustable cap 12 when the ends 11 are connected. The flexible wrap 10 has enough stretch to fit about the upper part of a patient's head securing there about just above the occipital ridges and ears of a patient. The stretchable flexible cap 12 applies minimal yet effective pressure around the head to stop bleeding. For larger heads, the cap may include an extra fold 12a around the rim of the cap 12, which may be folded down to form an extended rim 12b as shown in FIG. 1. This extra fold 12a, when folded against the cap 12, applies additional slight pressure around the rim of the cap 12 to hold the cap 12 in place without straps 14.

To secure the cap 12 in place, optional adjustable straps 14, 16 may be affixed to edges of the rim of the cap 12. Each strap 14, 16, has corresponding hook 15a and loop strips 15b affixed to their ends, 18, 20, which secure to one another to hold the cap 12 in place about the head. These adjustable straps 14, 16 hold the cap 12 anchored from under the chin about the head.

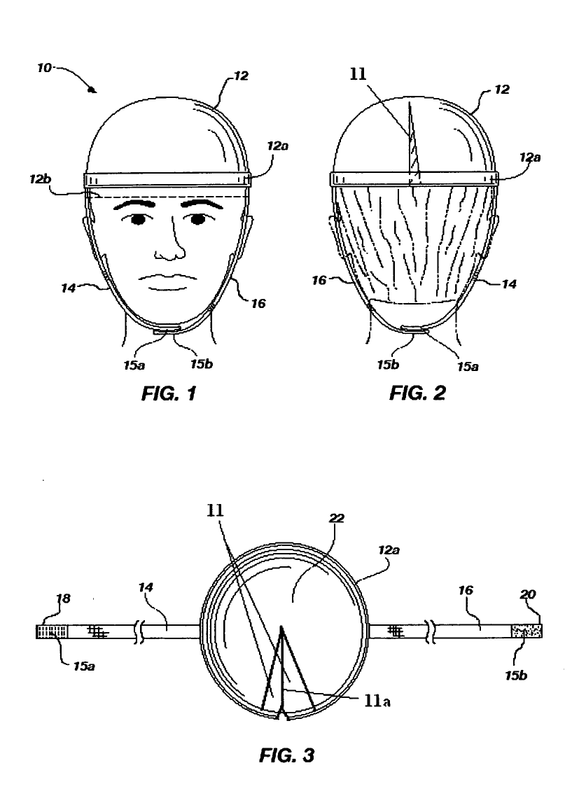

FIG. 2 is a rear view of the embodiment shown in FIG. 1 showing overlapping releaseably fastenable ends 11 removably secured to one another to form the cap 12. One preferred fastenable ends 11 employ corresponding hook and loop fasteners to removably secure the ends 12 together for this purpose.

On the inside of the adjustable cap 12 is attached a sterile dressing liner 22 as shown in FIG. 3. The stretchable adjustable cap 12 and liner 22 have enough stretch when placed on a patient's head to apply minimal pressure to control bleeding. It is adjustable to hold an icepack in place about the patent's head in closed dermal head injury (hematomas).

The adjustable cap 12 and sterile dressing liner 22 are preferably constructed of absorbent cotton, which stretches approximately 20% to apply minimal pressure on a head wound to stop bleeding, but have sufficient give to accommodate wound swelling, or to insert an ice pack over closed wounds to control swelling.

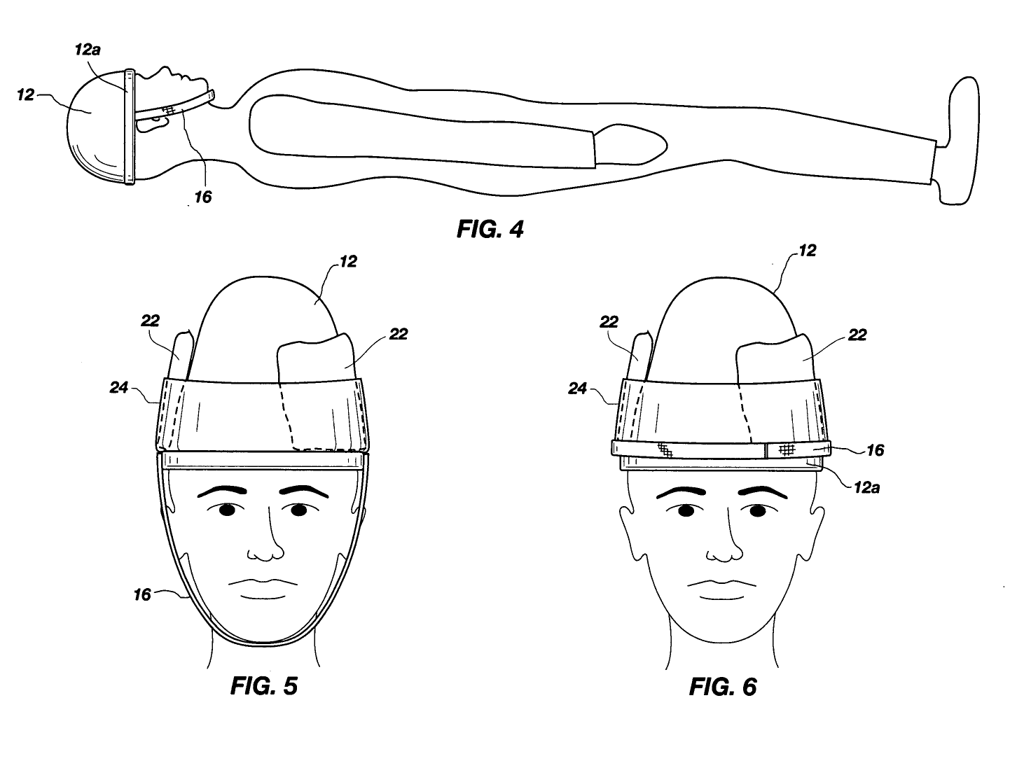

The invention 10 is readily wrapped about the head of a traumatized patient in the field to help maintain cervical spine immobilization as shown in FIG. 4. As the invention 10 is of one piece construction, it will not come apart during treatment or transport. It is fast and easy to apply to not only apply effective minimal pressure to the head wound to control the bleeding, but enables other treatment of the patient to be completed in a timely manner. If bleeding is profuse, additional dressings may be inserted beneath the adjustable cap to control bleeding.

FIG. 5 is a perspective of another preferred embodiment of the invention designed to carry ice packs 22 in an exterior pocket 24 affixed to the exterior of the adjustable cap 12. To secure the adjustable cap 12 and ice packs 22 in place, an optional removable circumferential adjustable strap 16 is affixed to edges of the cap with hook and loop strips (not shown). The adjustable strap 16 ends 18, 20 is then secured to the adjustable cap 12 above the fold 12a to hold the adjustable cap in place either about the head or under the chin.

Alternatively, this adjustable strap 16 enables the adjustable cap 12 to be secured in place above the eyes as shown in FIG. 6.

The embodiments of FIGS. 5 and 6 show a taller deeper adjustable cap 12, which can be positioned further down to cover a patient's entire face, if necessary, as long as airways are not compromised. This adjustable strap 16, when secured, may be tightened to place additional gentle pressure to hold the adjustable cap about the head to control bleeding from exterior wounds, where there are no intracranial injuries.

The above description and specification should not be construed as limiting the scope of the claims but as merely providing illustrations of some of the presently preferred embodiments of this invention. Thus, the claims themselves contain those features deemed essential to the invention.

We claim:

1. A head trauma bandage for covering the upper part of the head just above the occipital ridges and ears of a patient comprising:
a. a flexible stretchable bandage with a slit opening defined by opposite corresponding ends each with releasable connecting structure sized when the ends are connected to form a head opening to fit about and cover the upper part of the head just above the occipital ridges and ears of a patient with a head trauma; the ends affixed in a manner to apply minimal pressure to stop bleeding, and
b. a sterile dressing liner affixed inside of the stretchable bandage; said stretchable bandage and liner having enough stretch when placed on a patient to control bleeding and expand to accommodate intracranial (internal) swelling.

2. A head trauma bandage according to claim 1, wherein the stretchable bandage head opening about the head includes an extra fold, which may be folded down to form an extended bandage to cover more of the head in an unfolded mode.

3. A head trauma bandage according to claim 1, including a strap system associated with the stretchable bandage structured to secure the bandage to the head.

4. A head trauma bandage according to claim 3, including a plurality of opening fasteners placed about the head opening of the bandage, and wherein the strap system comprises a universal strap with first and second ends with corresponding openable and closable fasteners adapted to removably secure to the opening fasteners on opposite sides of the head opening of the cap after passing under the chin to secure the stretchable bandage to the patient's head, in one securing mode, and to secure to one another about the head to secure the stretchable bandage in a second securing mode; said straps of a length when secured to secure under the chin or about the head of the user.

5. A head trauma bandage for covering the upper part of the head just above the occipital ridges and ears of a patient with a head trauma comprising:
a. a flexible flat stretchable bandage with a slit opening defined by opposite corresponding ends each with releasable connecting structure sized when the ends are connected to form an adjustable cap with
i. a head opening to fit about and cover the upper part of the head just above the occipital ridges and ears of a patient with a head trauma; the corresponding ends affixed in a manner to apply minimal pressure to stop bleeding, and
ii. an extra fold, which may be folded down to form an extended cap to cover more of the upper part of the head in an unfolded mode;
b. a plurality of opening fasteners placed about the exterior of the cap proximate its opening,
c. a sterile dressing liner affixed to the interior of the cap; said cap and liner with enough stretch when placed on the head of a patient to apply minimal pressure to the head to control bleeding and accommodate swelling,
d. an exterior sleeve affixed to the exterior of the cap with pockets into which removable ice packs may be placed, and
e. a strap system associated with the cap to secure the cap and ice packs in place about the head.

6. A head trauma bandage according to claim 5, wherein the strap system comprises a universal strap with first and second ends with corresponding openable and closable fasteners adapted to removably secure to the opening fasteners on opposite sides of the opening of the cap after passing under the chin to secure the cap to the patient's head, in one securing mode, and to secure to one another about the head to secure the cap in a second securing mode; said straps of a length when secured to secure either under the chin or about the head of the user to hold the cap against the patient's head or secure ice packs in place about the head.

7. A head trauma bandage according to claim 5, wherein the strap system comprises a pair of straps with ends affixed on opposite sides of the cap opening with corresponding openable and closable fasteners adapted to secure the ends to one another under the chin to secure the cap about the head or about the top of the head to hold ice packs in place in a securing mode.

8. A head trauma cap bandage according to claim 6, wherein the fasteners comprise corresponding hook and loop strips.

9. A head trauma bandage according to claim 5, including a stretchable weather resistant cover affixed to cover the stretchable cap.

10. A head trauma bandage according to claim 5, wherein the cap is color coded and placed on the patient to indicate the severity of a patient's injuries.

11. A method of using a head trauma bandage for covering the upper part of a head wound just above the occipital ridges and ears of a patient comprising:
affixing over a traumatized patient's cranium
i. a flexible stretchable bandage with a slit opening defined by opposite corresponding ends each with releasable connecting structure sized when the ends are connected to form a head opening to fit about and cover the upper part of the head just above the occipital ridges and ears of a patient with a head trauma; the ends affixed in a manner to apply minimal pressure to stop bleeding, and
ii. a sterile dressing liner affixed inside of the stretchable bandage; said stretchable bandage and liner having enough stretch when placed on a patient to control bleeding and expand to accommodate swelling.

12. A method of using a head trauma bandage according to claim 11, including immobilizing the patient having head or spinal injuries first before applying the head trauma bandage.

13. A method of using a head trauma bandage according to claim 11, wherein the head trauma bandages is marked with different triage color codes to indicate the severity of a patient's injuries, and are selectively applied to a patient to indicate the type of medical response required.

14. A method of using a head trauma bandage according to claim 11, wherein the exterior of the bandage includes a sleeve with pockets sized to accommodate removable ice packs, which are selectively employed when needed to reduce swelling.

15. A method of using a head trauma bandage according to claim 11, including a strap system structured to removably secure the bandage to about the head.

16. A method of using a head trauma bandage according to claim 15, wherein the strap system comprises a pair of straps with first and second ends, the first ends attached to the opening of the cap, and the second ends having adjustable corresponding openable and closable fasteners; said straps of a length to secure to one another with the fasteners from under the chin or over the head; and securing the fastener's together to pull the cap from under the chin against the patient's head to apply additional minimal pressure to stop bleeding.

17. A method of using a head trauma bandage according to claim 15, wherein the strap system comprises a universal strap with first and second ends with corresponding openable and closable fasteners adapted to removably secure to the opening fasteners on opposite sides of the opening of the bandage after passing under the chin to secure the bandage to the patient's head, in one securing mode, and to secure to one another about the head to secure the bandage in a second securing mode; said straps of a length when secured to secure under the chin or about the head of the user to hold the cap against the patient's head or secure ice packs in place about the head.
